(19) United States
(12) Patent Application Publication
(10) Pub. No.: US 2026/0166262 A1
(43) Pub. Date: Jun. 18, 2026

(54) FOLEY CATHETER HAVING MEMBRANE

(71) Applicant: AP MDS Co., Ltd., Incheon (KR)

(72) Inventors: JONG JEONG KIM, Incheon (KR); Keon Wook WOO, Cheonan-si (KR); Myung Jae PARK, Incheon (KR)

(21) Appl. No.: 19/420,395

(22) Filed: Dec. 15, 2025

(30) Foreign Application Priority Data
Dec. 17, 2024 (KR) 10-2024-0188307

(51) Int. Cl. A61M 25/00

(52) U.S. Cl. CPC A61M 25/0017; A61M 25/0045; A61M 25/007; A61M 2210/1089

(57) ABSTRACT

The present invention relates to a membrane foley catheter including: a shaft composed of a material in which a carbon nanotube polymer formed by combining carbon nanotubes and zinc oxide is blended with silicone; a balloon composed of the material in which the CNT polymer is blended with silicone and is joined to the shaft so as to be inflatable by a first fluid introduced from an external source; and a membrane composed of the material in which the CNT polymer is blended with silicone, having a plurality of holes formed therein for discharging a second fluid introduced from an external source, and joined to the shaft.

### CROSS-REFERENCE TO RELATED APPLICATION

This application claims priority under 35 U.S.C § 119 to Korean Patent Application No. 10-2024-0188307 filed in the Korean Intellectual Property Office on Dec. 17, 2024, the entire contents of which are hereby incorporated by reference.

### BACKGROUND OF THE INVENTION

### Field of the Invention

The present invention relates to a foley catheter which is inserted in vivo.

### Description of the Prior Art

In general, patients with systemic or lower body paralysis caused by cerebral diseases such as stroke or spinal injuries are increasing year by year due to an increase in the elderly population and an increase rapidly in traffic accidents or industrial accidents.

A bladder paralysis is inevitably accompanied in such patients. A treatment of bladder paralysis is entirely dependent on the prognosis of the patient, and a foley catheter is maintained in the bladder as a treatment for these patients.

The foley catheter is made by attaching a foley to the distal end portion of the tubular shaft so that the foley is inflated by the fluid introduced from the outside to have a balloon shape, which the catheter is held in the bladder.

In the case of a conventional antibiotic catheter, an antibiotic drug or a substance is applied to a foley catheter made of a silicone to suppress invasion of bacteria. Although antibiotics are initially effective in antibiotics, biofilm formation is occurred inevitably due to the intubation of the urethra for more than from 2 to 3 days according to the nature of the catheter.

Such biofilm formation may cause the antibiotic effect of the foley catheter to decrease or disappear so that there is a problem that the complications such as urethritis is resulted and treatment for this should be accompanied and the length of hospital stay is prolonged.

In addition, since the antibiotic drugs or substances applied to the surface of the conventional foley catheter are always held in the fastening state, the urethritis and stones are formed, resulting in kidney failure in 40% of the total patients, which is the greatest cause of death.

In addition, in order to solve the problems of the above-mentioned antibiotic catheter, there are some products coated with antibacterial materials such as gold, silver or silver nano. However, there has been a problem that the antibacterial activity is decreased due to peeling of the coated antibacterial substance when used over a certain period of time.

Meanwhile, when a urethral stricture, which is narrowing of the urethral lumen, occurs due to factors such as inflammation or an immune response, a medication for treatment needs to be delivered using a foley catheter.

### SUMMARY OF THE INVENTION

A membrane foley catheter according to one embodiment of the present invention includes: a shaft composed of a material in which a carbon nanotube (CNT) polymer (CNT polymer) formed by combining carbon nanotubes (CNTs) and zinc oxide (ZnO) is blended with silicone; a balloon composed of the material in which the CNT polymer is blended with silicone and is joined to the shaft so as to be inflatable by a first fluid introduced from an external source; and a membrane composed of the material in which the CNT polymer is blended with silicone, having a plurality of holes formed therein for discharging a second fluid introduced from an external source, and joined to the shaft.

At least one of the balloon and the membrane may have a CNT polymer blending ratio different from a CNT polymer blending ratio of the shaft, and the membrane may have a CNT polymer blending ratio higher than a CNT polymer blending ratio of the shaft.

Meanwhile, the plurality of holes may be drug discharge holes for discharging a drug injected from an external source, and may be formed in the membrane by laser perforation.

Being constituting as the above, according to an embodiment of the present invention, a shaft, a balloon, and a membrane are made of a material in which a CNT polymer formed by combining CNTs and zinc oxide (ZnO) is blended with silicone so that the formation of a biofilm, which is the source of bacterial infection, can be inhibited without applying a separate antibiotic material.

According to another embodiment of the present invention, a plurality of holes for discharging a fluid such as a drug are formed by laser perforation in a catheter membrane made of a material in which a CNT polymer is blended with silicone, thereby facilitating the injection of a drug for treating a disease that may occur in the urethra, such as urethral stricture.

In addition, by maintaining induction of a biopotential effect of the CNT polymer uniformly in the balloon and the membrane, bacteria stenosed to the balloon and the membrane can be inactivated, there by maintaining antibacterial property.

In addition, according to still another embodiment of the present invention, there are no side effects such as resistance to antibiotics, the lifespan is determined by the electrostatic capacity of the CNT polymer, and the CNT polymer has high thermal conductivity which can minimize patient rejection during the process of inserting into the human body.

The effects of the present invention described above are merely one of various effects according to the present invention, and the present invention can be realized in various forms according to the application mode of the embodiment.

### BRIEF DESCRIPTION OF THE DRAWINGS

The combustion promoter according to the present invention will now be described by way of example only, with reference to the accompanying drawings, in which:

FIG. 1 is a diagram illustrating the configuration of a membrane foley catheter according to one embodiment of the present invention;

FIG. 2 is a cross-sectional view illustrating an example of the cross-sectional configuration of the membrane foley catheter illustrated in FIG. 1;

FIGS. 3 to 8 are diagrams illustrating experimental results on a biofilm formation inhibition effect of the material constituting the membrane foley catheter;

FIG. 9 is a diagram illustrating experimental results on a foreign body sensation reduction effect of the material constituting the membrane foley catheter upon insertion into the human body;

FIG. 10 is a diagram illustrating the configuration and operation of a membrane foley catheter according to one embodiment of the present invention;

FIGS. 11 to 13 are diagrams illustrating embodiments of the configuration of a balloon included in a membrane foley catheter according to the present invention;

FIGS. 14 to 17 are diagrams illustrating embodiments of the configuration of a membrane included in a membrane foley catheter according to the present invention;

FIG. 18 is a diagram illustrating the configuration of a membrane foley catheter according to another embodiment of the present invention; and

FIG. 19 is a cross-sectional view illustrating an example of the cross-sectional configuration of the membrane foley catheter illustrated in FIG. 18.

### DETAILED DESCRIPTION OF THE INVENTION

The following merely illustrates the principles of the present invention. Therefore, those skilled in the art will be able to implement the principles of the present invention and invent various devices encompassing the concept and scope of the present invention, even though not explicitly described or illustrated herein. Furthermore, it should be understood that all conditional terms and embodiments described herein are expressly intended solely to facilitate an understanding of the concepts of the present invention and are not intended to be limiting to the specifically recited embodiments and conditions.

In addition, all detailed descriptions enumerating specific embodiments, as well as principles, aspects, and embodiments of the present invention, should be understood to encompass structural and functional equivalents thereof. Furthermore, such equivalents should be understood to include not only currently known equivalents but also equivalents developed in the future, that is, all devices invented to perform the same function, regardless of structure.

In the claims of the present specification, a component expressed as a means for performing a function described in the detailed description is intended to include any method for performing the function, including, for example, a combination of circuit elements performing the function, or any form of software including firmware/microcode, and is combined with appropriate circuitry for executing the software to perform the function. The invention defined by these claims combines the functions provided by the various means enumerated and combined in the manner required by the claims, and therefore, any means capable of providing the functions should be understood to be equivalent to those disclosed herein.

The above-described objectives, features, and advantages will become more apparent through the following detailed description in conjunction with the accompanying drawings. Accordingly, those skilled in the art will be able to easily implement the technical spirit of the present invention. Furthermore, in describing the present invention, detailed descriptions of known technologies related to the present invention will be omitted when they are deemed to unnecessarily obscure the gist of the invention.

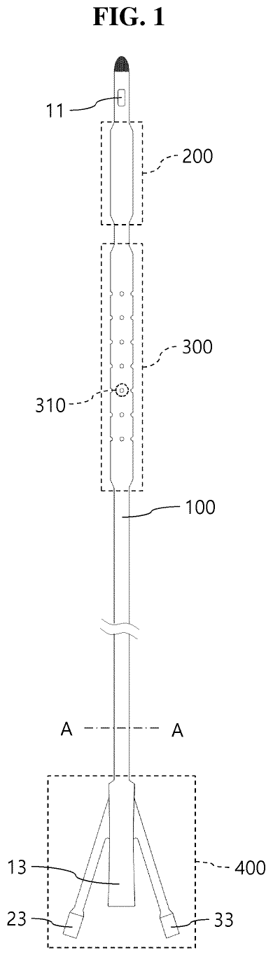

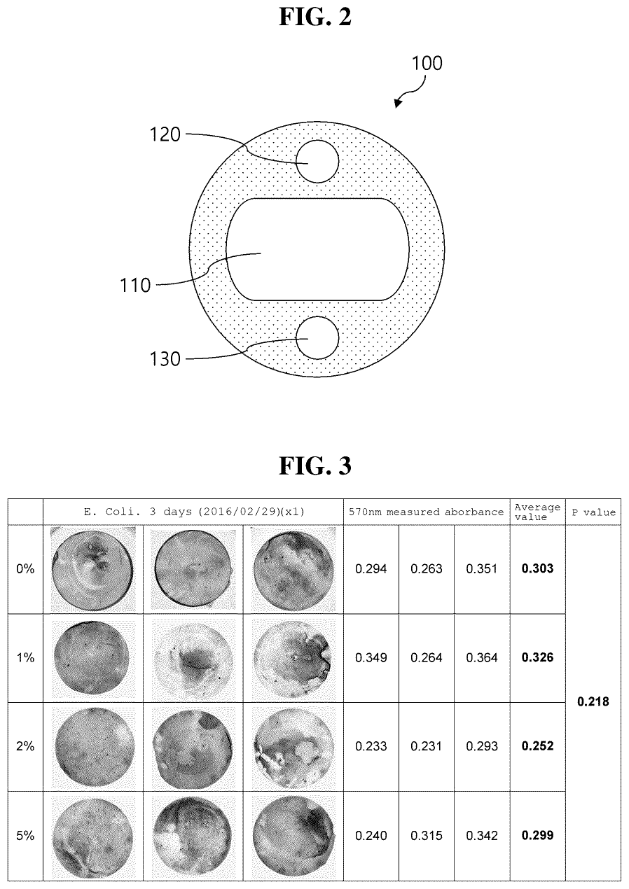

FIG. 1 illustrates the configuration of a membrane foley catheter according to one embodiment of the present invention, and FIG. 2 illustrates an example of a cross-sectional configuration of a membrane poly catheter, which is a cross-sectional view taken along line A-A shown in FIG. 1.

A catheter according to an embodiment of the present invention may be a urine catheter for discharging urine in a bladder of a patient and may be a foley catheter having a structure in which a urine inlet and an inflatable balloon are positioned at one end of a shaft, and the shaft is connected to a urine outlet positioned at the other end of the main body to discharge urine in the patient's bladder.

However, the catheter and the foley for catheter according to one embodiment of the present invention may be applied to various catheters such as a cardiovascular catheter in addition to the urethral catheter as described above.

Referring to FIGS. 1 and 2, a membrane foley catheter may include a shaft 100, which is a tubular body, a balloon 200 connected to the shaft 100 so as to be inflatable by a first fluid introduced from an external source, and a membrane 300 connected to the shaft 100, which has a plurality of holes formed therein for discharging a second fluid introduced from an external source.

The shaft 100 may have one end blocked and include passages formed therein, including a first passage 110, through which urine is discharged, a second passage 120, through which a first fluid is introduced and moved, and a third passage 130, through which a second fluid is introduced and moved.

A urine inlet 11 may be connected to the first passage 110 within the shaft 100, a fluid outlet (not shown) through which the first fluid is discharged to inflate the balloon 200 may be connected to the second passage 120, and a drug outlet (not shown) through which the second fluid is discharged for the injection of a drugs or the like may be connected to the third passage 130.

Meanwhile, at the other end of the shaft 100, a manifold 400 including a urine discharging portion 13, through which urine is discharged to the outside, a first fluid inlet 23, through which the first fluid is introduced from an external source, and a second fluid inlet 33, through which the second fluid is introduced from an external source, may be formed.

The urine discharging portion 13 is connected to the first passage 110 within the shaft 100, the first fluid inlet 23 is connected to the second passage 120, and the second fluid inlet 33 for injecting a drug or the like may be connected to the third passage 130.

In FIG. 2, a membrane foley catheter according to one embodiment of the present invention is described with an example in which three passages 110, 120, and 130 are formed within the shaft 100, but the present invention is not limited thereto.

The balloon 200 is composed of a material that may be stretched or expanded as the first fluid in introduced, and may be joined to the shaft 100 while surrounding the fluid outlet formed in the shaft 100.

Meanwhile, the membrane 300 may include a plurality of holes 310 for discharging a second fluid, for example, drug discharge holes through which a drug is discharged, and may be joined to the shaft 100 while surrounding the drug outlet formed in the shaft 100.

A fluid injected from an external source through the first inlet 23 may be delivered to a portion where the balloon 200 is joined through the second passage 120 and then flow out into a space between the balloon 200 and the shaft 100 through the fluid outlet formed in the shaft 100, thereby allowing the balloon 200 to inflate.

A drug injected from an external source through the second inlet 33 may be delivered to a portion where the membrane 300 is joined through the third passage 130, flow out into the space between the membrane 300 and the shaft 100 through a drug outlet formed in the shaft 100, and be discharged through the plurality of holes 310 formed in the membrane 300.

Here, the first fluid injected through the first inlet 23 may be a gas such as air or a liquid such as saline solution, and the second fluid injected through the second inlet 33 may be a drug for treating urethral stricture, but the present invention is not limited thereto.

The membrane foley catheter according to one embodiment of the present invention having the structures described with reference to FIGS. 1 and 2 are made of a material in which a carbon nanotube polymer (CNT polymer) is blended with silicone, and it is possible to inhibit the formation of a biofilm which is the source of bacterial infection, without applying a separate antibiotic.

The carbon nanotubes (CNTs) are cylindrical crystals made of a carbon atom and have a diameter from 2 to 20 nm and a length from several hundred to several thousand nm. One carbon atom in the CNT forms a hexagonal honeycomb pattern by sp2 bonding with three other carbon atoms around it, which is called nanotube because the diameter of the tube is very small of about nanometers (nm).

Furthermore, the CNT polymer is a polymer in which CNTs are combined with zinc oxide (ZnO), and wherein CNTs and zinc oxide (ZnO) may be polymerized at the same ratio or zinc oxide (ZnO) may have a higher ratio compared to CNTs, and vice versa as needed.

The shaft 100 included in the catheter according to an embodiment of the present invention may be composed of a material in which 100 parts by weight of silicone is blended with 1.0 to 2.2 parts by weight of the CNT polymer, but the blending ratio may be variable.

According to the present invention, the CNT polymer as a component of the shaft 100 has a constant capacitance in response to a potential in the intubated human body, so that such a capacitance is harmless to the human body, but has a galvanic effect which are deadly to bacteria and biofilms, which enable to minimizes the formation of biofilm, and the rejection of the subject during inserting process into the human body due to the high thermal conductivity which is characteristic of CNTs.

In addition, for the case of a catheter applied or coated with a conventional antibiotic, it is impossible to use the catheter for more than one week due to formation of biofilm and bacterial infection. But, the foley catheter according to the present invention can be used for at least 4 to 5 weeks due to the silicone which the CNT polymer having the above described effect is added.

According to an embodiment of the present invention, the CNT may be a multi-walled carbon nanotube (MWNT) since the multi-walled carbon nanotube (MWNT) has a merit to use in a solid state and is likely to be commercialized in terms of price.

As being described above, the foley catheter and the foley for catheter according to an embodiment of the present invention may each be composed of a material in which a CNT polymer formed by combining CNTs and zinc oxide (ZnO) is blended with silicone, as Chemical Formula 1 below.

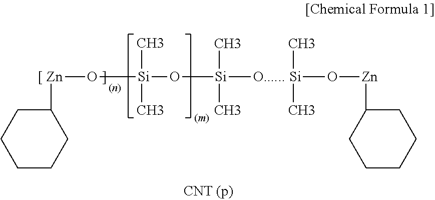

In Chemical Formula 1, m, n and p represent the number of each of molecules of silicone, zinc oxide (ZnO) and CNTs, m is 50 to 300, n is 7 to 30, and p is 10 to 50, but the present invention is not limited thereto.

Meanwhile, in the catheter and the foley for catheter, the above m, n, and p may be set different from each other.

In addition, the shaft 100 and the balloon for catheter 200 for constituting the catheter according to an embodiment of the present invention may be a tubular tube obtained by extruding a material in which a CNT polymer formed by combining CNTs and zinc oxide (ZnO) is blended with silicone, in a predetermined ratio.

Herein, the material may be formed by compounding CNTs and zinc oxide (ZnO) dispersed using a chemical vapor deposition (CVD) composite into silicone, for example, at a pressure of 1,000,000 Pa and a pressure of 50° C. for 30 minutes via dispersing process.

Accordingly, a CNT polymer composed of CNTs and zinc oxide (ZnO) may be uniformly inserted into the silicone, whereby the catheter and the foley for catheter made of this material can have antibacterial activity uniformly with regardless of position.

Hereinafter, the effect for inhibiting formation of biofilm at the material constituting the foley catheter and the foley for catheter according to one embodiment of the present invention will be described with reference to FIGS. 3 to 8.

FIGS. 3 to 5 show the results obtained by culturing E. coli, which is a major pathogen of urethritis, on a catheter slice composed of the above materials for 3 days, 5 days, and 7 days, and experiment of forming degree of biofilm using the crystal violet method.

With reference to FIG. 3, when E. coli was cultured for 3 days, the average value of absorbance was measured with 0.303 for the blending ratio of zinc oxide (ZnO) of 0%, the average value of absorbance was measured with 0.326 for the blending ratio of zinc oxide (ZnO) of 1%, the average value of absorbance was measured with 0.252 for the blending ratio of zinc oxide (ZnO) of 2%, and the average value of absorbance was measured with 0.299 for the blending ratio of zinc oxide (ZnO) of 3%.

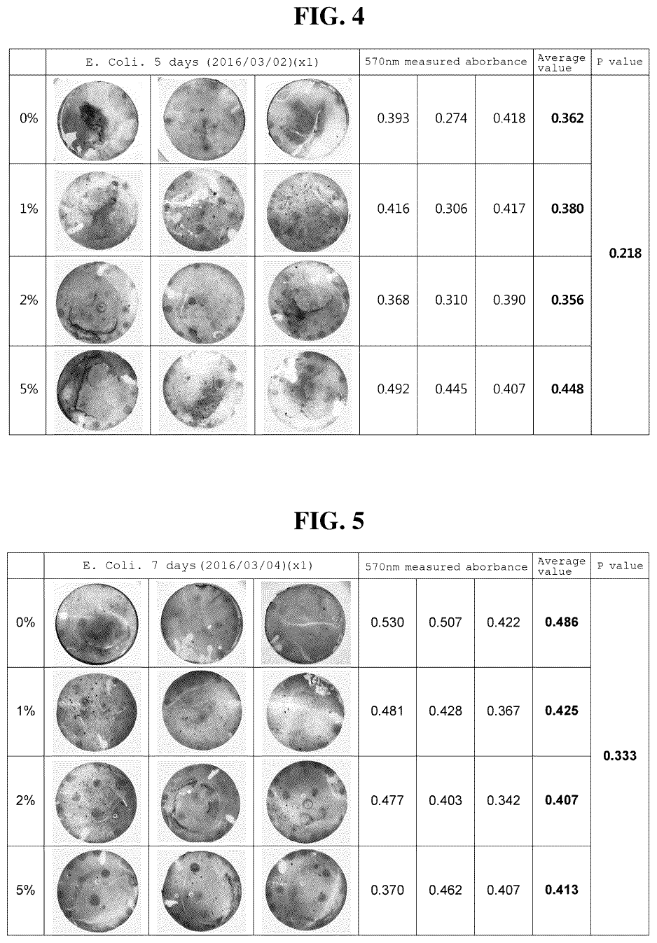

With reference to FIG. 4, when E. coli was cultured for 5 days, the average value of absorbance was measured with 0.362 for the blending ratio of zinc oxide (ZnO) of 0%, the average value of absorbance was measured with 0.380 for the blending ratio of zinc oxide (ZnO) of 1%, the average value of absorbance was measured with 0.356 for the blending ratio of zinc oxide (ZnO) of 2%, and the average value of absorbance was measured with 0.448 for the blending ratio of zinc oxide (ZnO) of 3%.

With reference to FIG. 5, when E. coli was cultured for 7 days, the average value of absorbance was measured with 0.486 for the blending ratio of zinc oxide (ZnO) of 0%, the average value of absorbance was measured with 0.425 for the blending ratio of zinc oxide (ZnO) of 1%, the average value of absorbance was measured with 0.407 for the blending ratio of zinc oxide (ZnO) of 2%, and the average value of absorbance was measured with 0.413 for the blending ratio of zinc oxide (ZnO) of 3%.

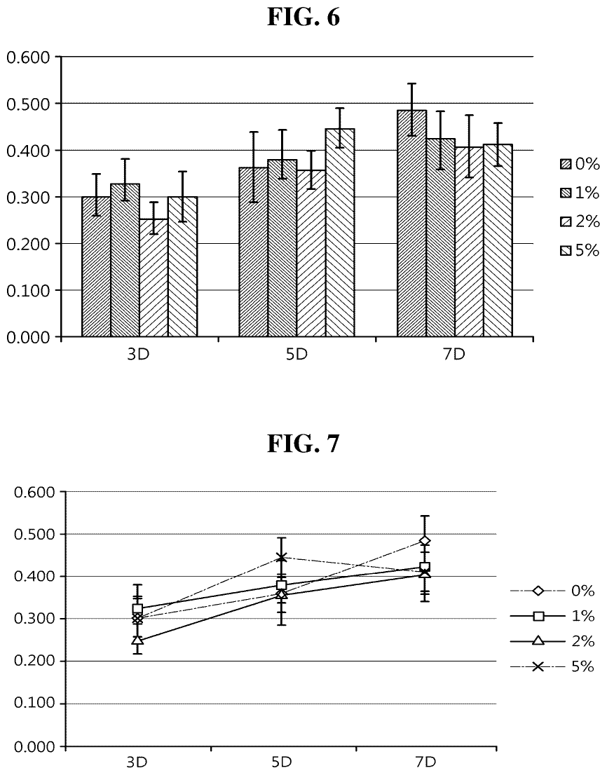

FIG. 6 is a graph showing the results of the experiment described above with respect to the experimental materials, and FIG. 7 is a graph showing the results of the experiments described above with respect to the culturing time.

According to the experimental results shown in FIGS. 3 to 6, for a material in which the silicone and the CNTs are blended with silicone (i.e. 0% of zinc oxide (ZnO)), the average value of absorbance is rapidly increased over time, thereby formation of the biofilm is increased.

Meanwhile, for a material in which 1% of zinc oxide (ZnO) is blended with silicone and CNTs, the average value of absorbance is slowly increased than that in the case of a material in which the CNTs are mixed with silicone (i.e. 0% of zinc oxide (ZnO)), thereby formation of the biofilm is suppressed to some extent.

In addition, for a material in which 2% of zinc oxide (ZnO) is blended with silicone and CNTs, the average value of absorbance is generally lower than that in the case of a material in which the CNTs are mixed with silicone (i.e. 0% of zinc oxide (ZnO)) and 1% of zinc oxide (ZnO) is mixed with silicone and CNTs, thereby the inhibitory effect on the biofilm formation of a major strain of urinary tract infection, E. coli (Escherichia coli) is clearly shown.

In addition, for material in which 5% of zinc oxide (ZnO) is blended with silicone and CNTs, the average value of absorbance after 7 days of culture was lower than before, resulting in an effect for inhibiting a biofilm formation according to use of long period of time.

Based on the above experimental results, when a catheter and a foley for catheter are composed of silicone and CNTs in which zinc oxide (ZnO) is blended with about 2%, an inhibitory effect on the biofilm formation of E. coli, which is a major pathogen of urethritis, can be stably achieved.

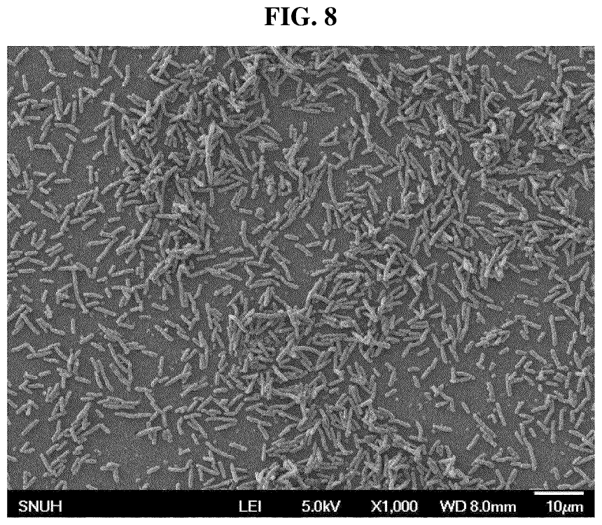

FIG. 8 is a photograph of the results obtained by culturing E. coli in a material having a zinc oxide (ZnO) blending ratio of 1% for 7 days and then taking the results of the experiment with a scanning electron microscope (SEM).

With reference to FIG. 8, even after 7 days of culture, it can be seen that the microorganism, E. coli, did not form a biofilm by aggregation.

In addition, referring to the experimental results shown in Table 1 below, the material constituting the catheter and the foley for catheter according to an embodiment of the present invention have a bactericidal reduction rate of more than 99.9% and a bacteriostatic reduction rate for Staphylococcus aureus, pneumococcus, Escherichia coli and Pseudomonas aeruginosa in addition to said E. coli.

TABLE 1

| | Staphylococcus aureus | | | | | |
|---|---|---|---|---|---|---|
| | (CFU/ml) | | Proliferation | Bactericidal | Bacteriostatic | |
| | Initial | 24 | value | reduction rate | reduction rate | Sample |
| NO | vaccination | hours | (F) | (%) | (%) | name |
| 1 | 3.1E+04 | 1.7E+06 | 55 | — | — | SD BLANK |
| 2 | | 1.0E+01 | | 99.97% | 99.99% | P-1460E 15 |
| 3 | | 1.0E+01 | | 99.97% | 99.99% | ZnO 15 |
| | Pneumococcus | | | | | |
| | (CFU/ml) | | Proliferation | Bactericidal | Bacteriostatic | |
| | Initial | 24 | value | reduction rate | reduction rate | Sample |
| NO | vaccination | hours | (F) | (%) | (%) | name |
| 1 | 2.2E+04 | 3.0E+06 | 136 | — | — | SD BLANK |
| 2 | | 1.0E+01 | | 99.95% | 99.99% | P-1460E 15 |
| 3 | | 1.0E+01 | | 99.95% | 99.99% | ZnO 15 |
| | Escherichia coli | | | | | |
| | (CFU/ml) | | Proliferation | Bactericidal | Bacteriostatic | |
| | Initial | 24 | value | reduction rate | reduction rate | Sample |
| NO | vaccination | hours | (F) | (%) | (%) | name |
| 1 | 2.1E+04 | 1.8E+06 | 86 | — | — | SD BLANK |
| 2 | | 1.0E+01 | | 99.95% | 99.99% | P-1460E 15 |
| 3 | | 1.0E+01 | | 99.95% | 99.99% | ZnO 15 |
| | Pseudomonas | | | | | |
| | aeruginosa | | | | | |
| | (CFU/ml) | | Proliferation | Bactericidal | Bacteriostatic | |
| | Initial | 24 | value | reduction rate | reduction rate | Sample |
| NO | vaccination | hours | (F) | (%) | (%) | name |
| 1 | 1.2E+04 | 1.8E+06 | 150 | — | — | SD BLANK |
| 2 | | 1.0E+01 | | 99.92% | 99.99% | P-1460E 15 |
| 3 | | 1.0E+01 | | 99.92% | 99.99% | ZnO 15 |

Meanwhile, the catheter and the foley for catheter configured as described above can minimize the patient's rejection feeling in the process of inserting the human body due to the high heat conduction characteristic of CNTs.

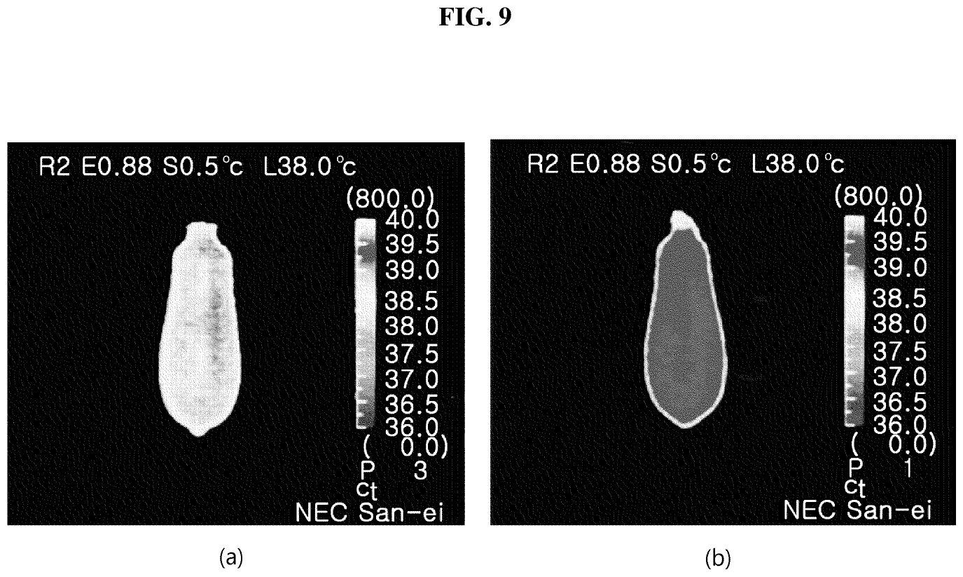

FIG. 9 shows experimental results for explaining an effect of reducing the foreign sensation when the materials constituting a foley catheter and a foley for catheter are inserted into the human body. FIG. 9A is a thermal image of a catheter made of silicone in which CNTs do not internalized, and FIG. 9B is a thermal image of a catheter made of silicone in which CNTs are internalized.

With reference to FIGS. 9A and 9B, it can be seen that the temperature distribution is uniform due to the high thermal conductivity in the case of a catheter composed of silicone in which CNTs are internalized.

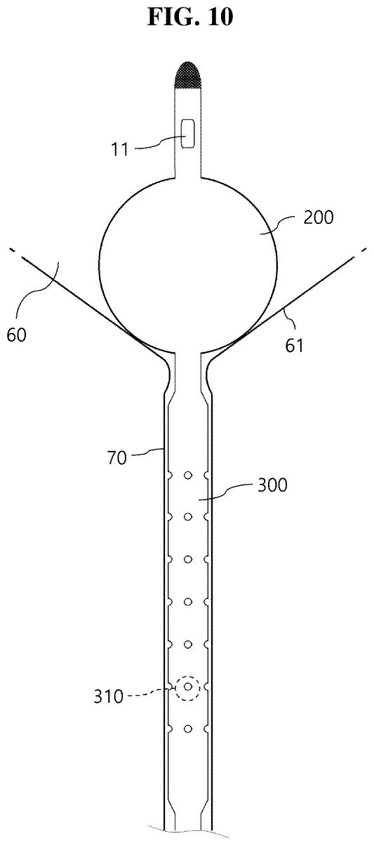

FIG. 10 is a drawing for explaining the configuration and operation of a membrane foley catheter according to an embodiment of the present invention. Descriptions of the same configurations as those described with reference to FIGS. 1 to 9 will be omitted herein.

Referring to FIG. 10, a shaft 100 is formed with a urine inlet 11 and a balloon 200 adjacent one end of the membrane foley catheter that is inserted into the bladder 60.

The balloon 200 may inflate to take the shape of a balloon when a first fluid is introduced from the first inlet 23 installed on the other side of the catheter.

For example, saline solution may be introduced by pre-injecting saline solution into a syringe, or the like, inserting a syringe needle into an inlet hole at an end of the fluid inlet 23, and compressing the syringe.

When the balloon 200 formed adjacent to one end of a shaft 100 is inserted into the bladder, as saline solution injected into the fluid inlet 23 flows into the balloon 200 through the second passage 120 and the fluid outlet, the balloon 200 inflates into a balloon shape and hangs over the bladder neck 61, thereby securing the catheter within the bladder 60.

Meanwhile, a first passage 110 connected to the urine inlet 11 is formed in a central portion of the shaft 100, and a urine discharging portion 13 is formed at an end of the first passage 110.

For example, urine may flow into the first passage 110 through the urine inlet 11 and then be discharged to the outside through a discharge hole at the end of the urine discharging portion 13 positioned outside the urethra 70.

In addition, as illustrated in FIG. 10, when the membrane foley catheter is inserted into the human body, the membrane 300 may be joined to a predetermined position of the shaft 100 so as to be positioned at the urethra 70 area, and accordingly, the balloon 200 may be positioned between the urine inlet 110 and the membrane 300.

For example, when the membrane 300 is positioned within the urethra 70 and tightly attached thereto, a drug for treating urethral stricture injected into the second inlet 33 may flow into the membrane 300 through the third passage 130 and the drug outlet, and the drug flowing into the membrane 300 may be discharged into the urethra 70 through a plurality of drug discharge holes 310.

According to one embodiment of the present invention, a plurality of drug discharge holes 310 may be formed in the membrane 300 by laser perforation.

For example, the laser used to form the drug discharge holes 310 in the membrane 300 may have a wavelength ranging from 200 nm to 500 nm or a pulse width of 0 to 10 nsec, but the present invention is not limited thereto.

Meanwhile, as described above, the shaft 100, balloon 200, and membrane 300 may be composed of a material in which a CNT polymer formed by combining CNTs and zinc oxide (ZnO) is blended with silicone.

According to one embodiment of the present invention, the CNT polymer blending ratio of at least one of the balloon 200 and the membrane 300 may differ from the CNT polymer blending ratio of the shaft 100.

For example, the CNT polymer blending ratio of the membrane 300 may be higher than the CNT polymer blending ratio of the shaft 100.

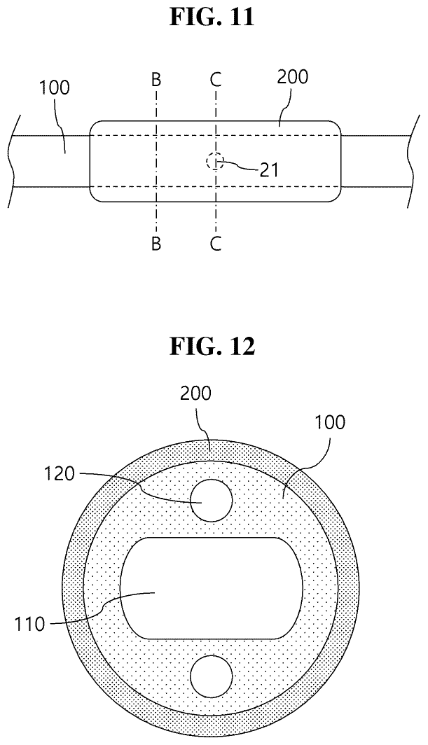

Hereinafter, embodiments of the membrane foley catheter according to the present invention will be described in more detail with reference to FIGS. 11 to 17.

FIGS. 11 to 13 are diagrams illustrating embodiments of the configuration of a balloon included in a membrane catheter foley according to the present invention. Descriptions of the same configurations of the balloon 200 with reference to FIGS. 1 to 10 will be omitted.

Referring to FIG. 11, the balloon 200 has joint surfaces formed at both ends for joining to the shaft 100. During the process of joining the balloon 200 to the shaft 100, the joint surfaces may be bonded to the shaft 100 using an adhesive.

As described above, the shaft 100 may be produced by extruding a material in which a CNT polymer formed by combining CNTs and zinc oxide (ZnO) is blended with silicone into a tubular body.

In addition, the material in which a CNT polymer formed by combining CNTs and zinc oxide (ZnO) is blended with silicone may be extruded or injected into a tubular body to produce the balloon 200.

Here, the blending ratio of CNTs, zinc oxide (ZnO), or CNT polymer used to produce the shaft 100 may differ from the blending ratio of carbon CNTs, zinc oxide (ZnO), or CNT polymer used to produce the balloon 200.

For example, when the balloon 200 inflates due to a fluid introduced from an external source, the surface area of the balloon increases, which may reduce the distribution of the CNT polymer per unit area, potentially reducing antibacterial activity.

As described above, when the balloon 200 inflates and antibacterial activity decreases, even when biofilm formation is suppressed on the shaft 100, a biofilm may be formed on the balloon 200 with reduced antibacterial activity, potentially causing bacterial infection.

To prevent this, the CNT polymer blending ratio of the balloon 200 is preferably higher than that of the shaft 100, and accordingly, even when the balloon 200 inflates, the distribution of the CNT polymer per unit area remains the same or similar to that of the shaft 100, the membrane foley catheter can have uniform antibacterial activity regardless of position.

For example, assuming that the balloon 200 inflates and the surface area thereof increases approximately 4-fold to 6-fold, when the shaft 100 is composed of a material in which 1.0 to 2.2 parts by weight of the CNT polymer is blended with 100 parts by weight of silicone, the balloon 200 may be composed of a material in which 4.0 to 13.2 parts by weight of the CNT polymer is blended per 100 parts by weight of silicone, in proportion to the increase in the surface area.

Meanwhile, in the case of the balloon 200, the values of n and m in Chemical Formula 1 may increase in proportion to the increase in the surface area during inflation of the balloon 200.

Once the shaft 100 and the balloon 200 are prepared, the joint surfaces at both ends of the balloon 200 may be bonded to the shaft 100, and a tip equipped with a urine inlet 11 may be formed at one end of the shaft 100.

FIG. 12 is a cross-sectional view taken along line B-B of the balloon 200 illustrated in FIG. 11. The balloon 200 may surround the shaft 100, which has a first passage 110 through which urine flows and a second passage 120 through which saline solution for inflating the balloon 200 flows.

In this structure, as described above, when the balloon 200 is manufactured using a material having a higher CNT polymer content than that of the shaft 100, a current may flow until the potential difference between the shaft 100 and the balloon 200 disappears.

This is because zinc oxide (ZnO) creates a high potential, causing a current to flow from the balloon 200 to the shaft 100 at a contact point. When the potential difference between the shaft 100 and the balloon 200 disappears, the capacitance of the balloon 200 during inflation may decrease, resulting in a decrease in antibacterial activity.

To prevent a current from flowing from the balloon 200 to the shaft 100, an insulating layer (not shown) may be formed between the shaft 100 and the balloon 200 for catheter.

The insulating layer may be composed of gas, such as an air layer or a sterilizing gas layer (e.g., ethylene oxide (EO) gas), or a high-concentration CNT coating layer.

By preventing a current from flowing from the balloon 200 to the shaft 100 by the insulating layer, the potential difference between the shaft 100 and the balloon 200, which varies depending on the CNT polymer blending ratio, can be maintained, and as a result, antibacterial activity of the balloon 200 can be maintained within a range similar to or the same to that of the shaft 100, even when the balloon 200 inflates.

As described above, in order to form an insulating layer between the shaft 100 and the balloon 200, an air layer may be injected between the shaft 100 and the balloon 200 during the formation process of the balloon 200, or a step of treating an outer surface of the shaft 100 with an EO gas or applying high-concentration CNTs may be added.

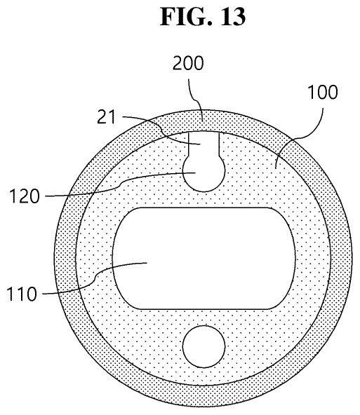

FIG. 13 is a cross-sectional view taken along the C-C line of the balloon 200 illustrated in FIG. 11, in which a fluid outlet 21 is formed in the shaft 100, so that a fluid (e.g., saline solution) moving through the second passage 120 may flow into the space between the balloon 200 and the shaft 100 through the fluid outlet 21, thereby inflating the balloon 200.

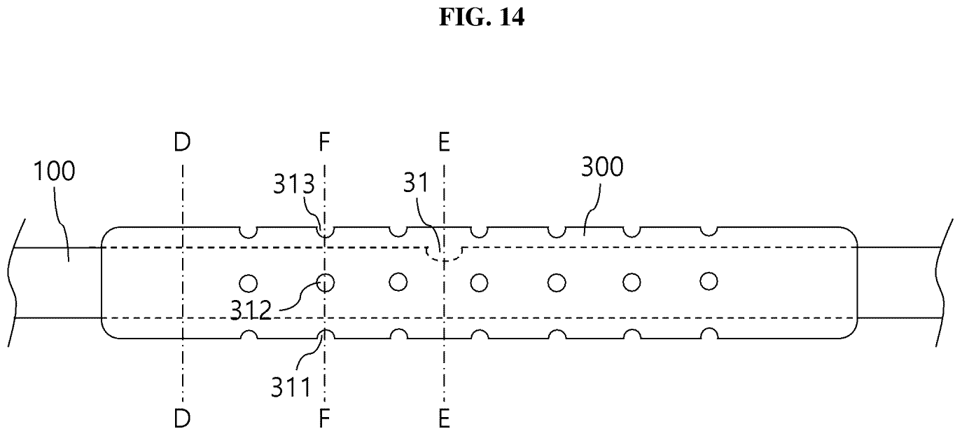

FIGS. 14 to 17 illustrate embodiments of the membrane configuration provided in the membrane catheter foley according to the present invention. Descriptions of the same configurations of the membrane 300 illustrated with reference to FIGS. 1 to 13 will be omitted.

Referring to FIG. 14, joint surfaces are formed at both ends of the membrane 300 for joining to the shaft 100. During the process of joining the membrane 300 to the shaft 100, the joint surfaces may be bonded to the shaft 100 using an adhesive.

As described above, the material in which a CNT polymer formed by combining CNTs and zinc oxide (ZnO) is blended with silicone may be extruded or injected into a tubular body to produce the membrane 300.

In addition, a drug outlet 31 may be formed in the shaft 100 to introduce a drug injected from an external source into the membrane 300, and a plurality of drug discharge holes 311, 312, and 313 may be formed in the membrane 300 by laser perforation.

Here, the size of the drug discharge hole formed in the membrane 300 is very small compared to the size of the drug discharge hole 31 formed in the shaft 100, and accordingly, the drug introduced into the membrane 300 may be discharged into the urethra 70 through the drug discharge holes while maintaining the pressure inside the membrane 300 within a certain range.

Meanwhile, the blending ratio of CNTs, zinc oxide (ZnO), or CNT polymer used to produce the shaft 100 may differ from the blending ratio of CNTs, zinc oxide (ZnO), or CNT polymer used to produce the membrane 300.

For example, when the drug discharge holes 311, 312, and 313 are blocked while the shaft 100 and the membrane 300 are in close contact, the drug injected into the second inlet 33 may not be discharged into the urethra 70 through the drug discharge holes 311, 312, and 313.

To prevent this, the CNT polymer blending ratio of the membrane 300 may preferably be higher than that of the shaft 100. In this case, the gap between the shaft 100 and the membrane 300 is maintained by the galvanic effect so that the drug discharge holes 311, 312, and 313 formed in the membrane 300 may not be blocked.

For example, the membrane 300 may be composed of a material having the same CNT polymer blending ratio as the balloon 200. In this case, when the shaft 100 is composed of a material in which 1.0 to 2.2 parts by weight of the CNT polymer is blended with 100 parts by weight of silicone, the membrane 300 may be composed of a material in which 4.0 to 13.2 parts by weight of the CNT polymer is blended with 100 parts by weight of silicone.

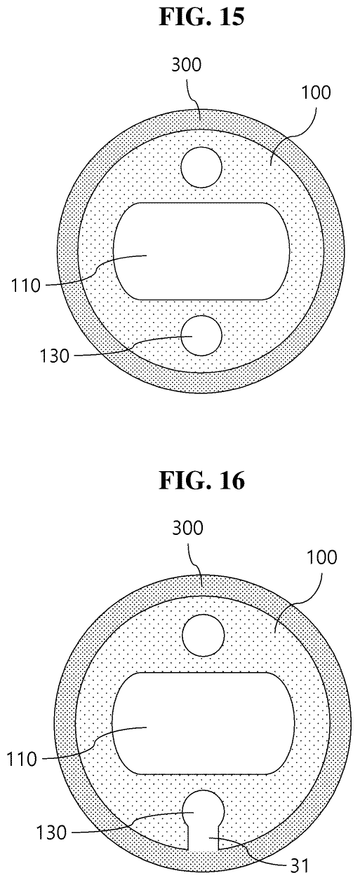

FIG. 15 is a cross-sectional view taken along the D-D line of the membrane 300 illustrated in FIG. 14. The membrane 300 may surround the shaft 100 including a third passage 130 formed therein through which a drug discharged into the urethra 70 moves through the membrane 300.

In this structure, as described above, when the membrane 300 is manufactured using a material having a higher CNT polymer content than that of the shaft 100, a current may flow until the potential difference between the shaft 100 and the membrane 300 disappears.

This is because zinc oxide (ZnO) creates a high potential, causing a current to flow from the membrane 300 to the shaft 100 at a contact point. When the potential difference between the shaft 100 and the membrane 300 disappears, the gap between the shaft 100 and the membrane 300 narrows, potentially blocking the drug discharge holes 311, 312, and 313.

To prevent a current from flowing from the membrane 300 to the shaft 100, an insulating layer (not shown) may be formed between the shaft 100 and the membrane 300.

The insulating layer may be composed of gas such as an air layer or a sterilizing gas layer (e.g., EO gas), or may be composed of a high-concentration CNT coating layer.

By preventing a current from flowing from the membrane 200 to the shaft 100 by the insulating layer, the potential difference between the shaft 100 and the membrane 300 can be maintained according to the difference in the CNT polymer blending ratio.

As described above, in order to form an insulating layer between the shaft 100 and the membrane 300, an air layer may be injected between the shaft 100 and the membrane 300 during the formation process of the membrane 300, or a step of treating an outer surface of the shaft 100 with an EO gas or applying high-concentration CNTs may be added.

FIG. 16 is a cross-sectional view taken along the line E-E of the membrane 300 illustrated in FIG. 14, in which a drug outlet 31 is formed in the shaft 100, so that a drug moving through the third passage 130 may flow into the space between the membrane 300 and the shaft 100 through the drug outlet 31.

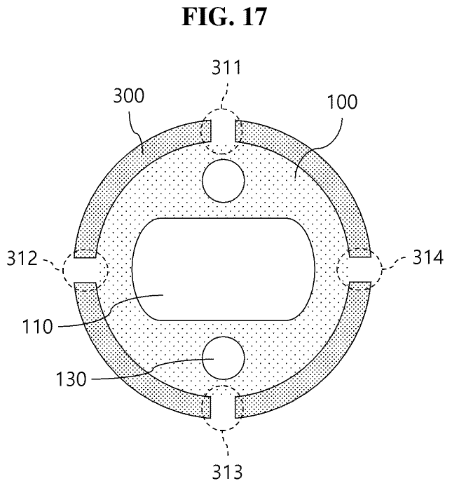

FIG. 17 is a cross-sectional view taken along the line F-F of the membrane 300 illustrated in FIG. 14, in which a drug flowing into the space between the membrane 300 and the shaft 100 may be discharged into the urethra 70 through a plurality of drug discharge holes 311 to 314.

Although the membrane foley catheter according to the present invention has been described above as a three-way catheter having three passages 110, 120, and 130, the present invention is not limited thereto and may also be applied to a membrane foley catheter having four or more passages.

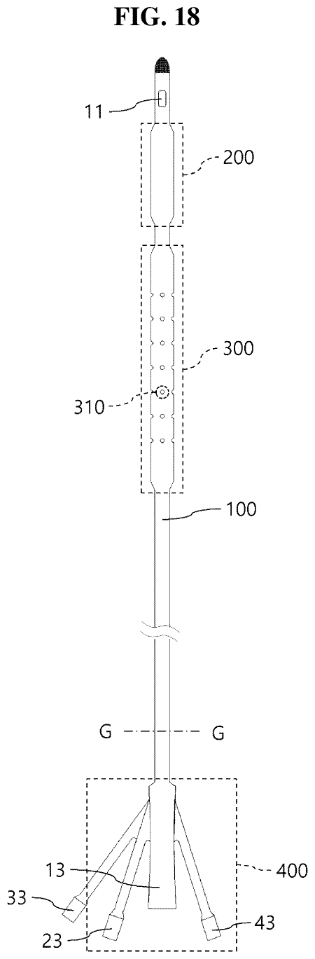

FIG. 18 is a diagram illustrating the configuration of a membrane foley catheter according to another embodiment of the present invention. Descriptions of the same configurations as those described with reference to FIGS. 1 to 17 will be omitted.

Referring to FIG. 18, the manifold 400 formed at the other end of the shaft 100 may include a urine discharging portion 13 for discharging urine to the outside, a fluid inlet 23 for introducing a first fluid (e.g., saline solution) from an external source to inflate the balloon 200, a drug injection portion 33 for injecting a second fluid (e.g., a drug) from an external source, and a cleaning solution injection portion 43 for injecting a third fluid (e.g., a cleaning solution) from an external source.

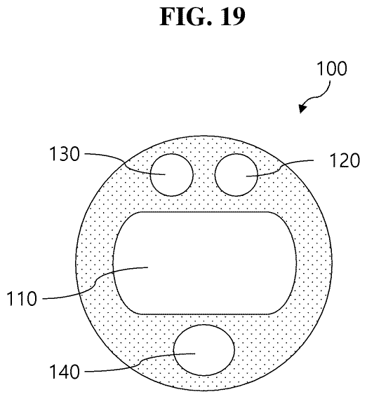

FIG. 19 is a cross-sectional view taken along the G-G line of the shaft 100 illustrated in FIG. 18.

Referring to FIG. 19, within the shaft 100, a first passage 110 through which urine moves to be discharged, a second passage 120 through which a first fluid (e.g., saline solution) for inflating the balloon 200 is introduced and moves, a third passage 130 through which a second fluid (e.g., a drug) is injected and moves, and a fourth passage 140 through which a third fluid (e.g., a cleaning solution) is injected and moves.

As described above, the urine inlet 11 is connected to the first passage 110 within the shaft 100, and the fluid outlet 21 through which the first fluid (e.g., saline solution) flows out to inflate the balloon 200 may be connected to the second passage 120, and the drug outlet 31 through which the second fluid (e.g., drug) flows out may be connected to the third passage 120.

Meanwhile, the urine inlet 11 may be connected to the fourth passage 140 within the shaft 100 so that the third fluid (e.g., cleaning solution) moving through the fourth passage 140 may be discharged through the urine inlet 11.

For example, a bladder cleaning solution injected into the cleaning solution injection unit 24 may move through the fourth passage 140 and be discharged into the bladder 60 through the urine inlet 11.

In the above, embodiments of the present invention have been described by taking as an example the membrane foley catheter according to the present invention for discharging urine and injecting a drug for treating urethral stricture or a bladder washing solution, but the present invention is not limited thereto, and may be applied to various types of catheters, such as cardiovascular catheters, drugs, washing solutions, and the like.

Although the preferred embodiments of the present invention have been illustrated and described above, the present invention is not limited to the above-described specific embodiments, and various modifications can be made by those skilled in the art without departing from the gist of the present invention as claimed in the claims. Furthermore, such modifications should not be understood individually from the technical spirit or prospect of the present invention.

What is claimed is:

1. A membrane foley catheter which is characterized in that the foley catheter comprises a shaft composed of a material in which a carbon nanotube (CNT) polymer (CNT polymer) formed by combining carbon nanotubes (CNTs) and zinc oxide (ZnO) is blended with silicone;
a balloon composed of the material in which the CNT polymer is blended with silicone and is joined to the shaft so as to be inflatable by a first fluid introduced from an external source; and
a membrane composed of the material in which the CNT polymer is blended with silicone, having a plurality of holes formed therein for discharging a second fluid introduced from an external source, and joined to the shaft.

2. The membrane foley catheter of claim 1, wherein at least one of the balloon and the membrane has a CNT polymer blending ratio different from a CNT polymer blending ratio of the shaft.

3. The membrane foley catheter of claim 2, wherein the membrane has a CNT polymer blending ratio higher than a CNT polymer blending ratio of the shaft.

4. The membrane foley catheter of claim 2, wherein the plurality of holes are drug discharge holes for discharging a drug injected from an external source.

5. The membrane foley catheter of claim 1, wherein the plurality of holes are formed in the membrane by laser perforation.

6. The membrane foley catheter of claim 1, further comprising a urine inlet formed at one end of the shaft for allowing urine to flow into the shaft, wherein the balloon is positioned between the urine inlet and the membrane.

7. The membrane foley catheter of claim 1, wherein the membrane is joined to a predetermined position of the shaft so that the membrane is positioned at a urethra region when inserted into a human body.

8. The membrane foley catheter of claim 1, wherein, in the shaft, a first passage for discharging urine, a second passage for introducing the first fluid, and a third passage for introducing the second fluid are formed.

9. The membrane foley catheter of claim 8, further comprising a drug outlet formed in the third passage for discharging the second fluid into a space between the shaft and the membrane.

10. The membrane foley catheter of claim 8, wherein, in the shaft, a fourth passage is further formed for movement of a third fluid introduced from an external source.

11. The membrane foley catheter of claim 1, wherein an insulating layer is formed between the membrane and the shaft.

12. The membrane foley catheter of claim 11, wherein the insulating layer is composed of an air layer or a sterilizing gas layer.

13. The membrane foley catheter of claim 11, wherein the insulating layer is composed of a CNT coating layer.
